(19) United States
(12) Patent Application Publication
(10) Pub. No.: US 2005/0186602 A1
(43) Pub. Date: Aug. 25, 2005

(54) Method for amplifying a nucleic acid using a solid phase material coated with a carboxyl group or amino group

(76) Inventors: Joon-ho Kim, Seongnam-si (KR); Yoon-kyoung Cho, Suwon-si (KR); Jung-joo Hwang, Suwon-si (KR); Geun-bae Lim, Pohang-si (KR); Jeong-gun Lee, Seoul (KR)

Correspondence Address: CANTOR COLBURN, LLP, 55 GRIFFIN ROAD SOUTH, BLOOMFIELD, CT, 06002, US

(21) Appl. No.: 11/042,376

(22) Filed: Jan. 25, 2005

(30) Foreign Application Priority Data
Jan. 28, 2004 (KR) 2004-0005503

(51) Int. Cl. C12Q 1/68; C12P 19/34

(52) U.S. Cl. 435/6; 435/91.2

(57) ABSTRACT

A method for amplifying a nucleic acid using a solid phase material coated with a carboxyl group or an amino group is provided. The method includes contacting a mixture of a nucleic acid containing sample and a salt solution with the solid phase material coated with a carboxyl group or an amino group to form a nucleic acid-solid phase material complex, washing the nucleic acid-solid phase material complex with a wash buffer, and adding a reaction solution for amplifying a nucleic acid to the nucleic acid-solid phase material complex to perform an amplification reaction.

### BACKGROUND OF THE INVENTION

This application claims the benefit of Korean Patent Application No. 10-2004-0005503, filed on Jan. 28, 2004, in the Korean Intellectual Property Office, the disclosure of which is incorporated herein in its entirety by reference.

1. Field of the Invention

The present invention relates to a method for amplifying a nucleic acid using a solid phase material coated with a carboxyl group or an amino group.

2. Description of the Related Art

Methods for isolating nucleic acids using solid phase materials are known in the art. For example, U.S. Pat. No. 5,234,809 issued to Boom discloses a method for isolating a nucleic acid using a solid phase material to which the nucleic acid may bind. The method comprises mixing a starting material containing nucleic acids, a chaotropic material and a nucleic acid binding solid phase material, forming a solid phase material-nucleic acid complex and eluting a nucleic acid from the complex to separate a nucleic acid. The method further comprises adding a mixture containing a component capable to amplifying a nucleic acid to the solid phase material-nucleic acid complex, and eluting the nucleic acid from the solid phase material to amplify the nucleic acid. Examples of the chaotropic material include quanidinium salts, sodium iodide, sodium thiocyanate, and urea. Examples of the solid phase include silica, and polystyrene latex.

However, the method requires the use of the chaotropic material. Without the chaotropic material, the nucleic acid cannot bind to the solid phase materia. In addition, the chaotropic material is harmful to humans and must be removed during the isolation or from the nucleic acids after the isolation.

U.S. Pat. No. 6,291,166 (Xtrana) discloses a method for archiving a nucleic acid using a solid phase matrix. The method includes irreversibly binding a nucleic acid to a solid phase matrix, wherein the solid phase matrix is characterized by an electropositive material rendered hydrophilic. The solid phase matrix may consist of silicon (Si), boron (B) or aluminum (Al). The electropositive material may be rendered hydrophilic using a basic solution, such as an NaOH solution. The nucleic acid irreversibly bound to the solid phase matrix in this method can be amplified by a method for amplifying a nucleic acid, such as PCR, SDA, and NASBA.

In this method, the nucleic acid irreversibly binds to the solid phase matrix and thus, amplification is carried out with the nucleic acid bound to the solid phase material. However, to amplify a nucleic acid, the nucleic acid must be separated in single strands. Thus, amplification efficiency is very low.

U.S. Pat. No. 5,898,071 discloses a method of non-specifically and reversibly binding nucleic acids to magnetic microparticles having a surface coated with a functional group. Specifically, the method includes combining magnetic microparticles whose surfaces have bound thereto a functional group which reversibly binds polynucleotide and a solution containing polynucleotides and adjusting the concentrations of salt and polyethylene glycol (PEG) in the obtained mixture to bind the polynucleotide onto the surfaces of the magnetic microparticles. The magnetic microparticles may be magnetic microparticles coated with carboxyl groups. However, this method has a disadvantage that the magnetic particles should be used.

The present inventors conducted research on a method for isolating a nucleic acid based on the conventional methods and discovered a method in which a nucleic acid can reversibly bind to a substrate coated with a carboxyl group or an amino group.

### SUMMARY OF THE INVENTION

The present invention provides a method for amplifying a nucleic acid on the solid material used in isolating the nucleic acid.

According to an aspect of the present invention, there is provided a method for amplifying a nucleic acid using a solid phase material coated with a carboxyl group or an amino group, comprising: contacting a mixture of a nucleic acid containing sample and a salt solution with the solid phase material coated with a carboxyl group or an amino group to form a nucleic acid-solid phase material complex; washing the nucleic acid-solid phase material complex with a wash buffer; and adding a reaction solution for amplifying a nucleic acid to the nucleic acid-solid phase material complex to perform an amplification reaction.

### BRIEF DESCRIPTION OF THE DRAWINGS

The above and other features and advantages of the present invention will become more apparent by describing in detail exemplary embodiments thereof with reference to the attached drawings in which:

FIG. 1 is a view illustrating results of gel electrophoresis analysis for DNA isolated according to an embodiment of the present invention;

FIGS. 2A and 2B are views illustrating results of a real-time PCR in which a HBV plasmid DNA isolated by a method according to an embodiment of the present invention was used as a template;

FIG. 3 is a view illustrating the effect of a PEG contained in a binding buffer on the isolation of DNA by a method according to an embodiment of the present invention.

FIG. 4A is a view illustrating results of a real-time PCR using the DNA products as a template, the DNA products obtained by using the initial DNA concentrations of 10³, 10⁵ and 10⁷ copies/μl, respectively;

FIG. 4B is a view illustrating a threshold cycle versus the initial DNA concentrations in FIG. 4A;

FIGS. 5A and 5B are schematic views illustrating a polymer chamber used in an embodiment of the present invention and the procedure of mounting the polymer chamber to a PCR apparatus; and

FIG. 6 is a view illustrating the results of gel electrophoresis analysis for a PCR product amplified by a PCR on a glass substrate coated with a carboxyl group.

### DETAILED DESCRIPTION OF THE INVENTION

According to an embodiment of the present invention, there is provided a method for amplifying a nucleic acid using a solid phase material coated with a carboxyl group or an amino group, comprising:
contacting a mixture of a nucleic acid containing sample and a salt solution with the solid phase material coated with a carboxyl group or an amino group to form a nucleic acid-solid phase material complex;
washing the nucleic acid-solid phase material complex with a wash buffer; and
adding a reaction solution for amplifying a nucleic acid to the nucleic acid-solid phase material complex to perform an amplification reaction.

In an embodiment of the present invention, the nucleic acid containing sample may be a biological material. Examples of the biological sample include blood, serum, buffy coat, urine, feces, cerebrospinal fluid, sperm, saliva, tissues, and cell cultures. The nucleic acid containing sample may be a non-biological material containing a nucleic acid. For the biological sample, if an obstacle, such as cell wall, cell membrane and envelope prevents a direct contact of the nucleic acid with a surface of the solid phase material coated with a carboxyl group or an amino group, a pretreatment can be performed with a substance that can kill a cell, such as a detergent or an organic solvent. For example, a cell may be ruptured using NaOH and made neutral, and then the solvent may be replaced by a salt, such as NaCl, solution used in an embodiment of the present invention, for a subsequent purification.

The salt solution may be a solution containing at least one salt selected from the group consisting of NaCl, MgCl₂, KCl, and CaCl₂. The salt may be contained at a concentration of 0.5 to 5 M.

In an embodiment of the present invention, the solid phase material may be any solid phase material coated with a carboxyl group or an amino group. Examples of the solid phase material include, but not are limited to, glass, silicon, and plastic materials, such as polyethylene, polypropylene, and polyacrylamide. Preferably, the solid phase material is glass. The solid phase material coated with a carboxyl group or an amino group used in the embodiment of the present invention may be prepared, for example, by coating a slide glass with GAPA (y-aminopropyltriethoxy silane) by a dipping method to obtain a substrate coated with an amino group, and then coating the substrate with succinic anhydride by the dipping method to obtain the substrate further coated with a carboxyl group.

In an embodiment of the present invention, the washing operation may be carried out with a wash buffer containing ethanol and EDTA. The wash buffer may be an aqueous solution containing 70% of ethanol and 10 mM EDTA.

In the method for amplifying a nucleic acid according to an embodiment of the present invention, the amplification may be carried out using various amplification methods known in the art. Examples of amplification methods include, but are not limited to, PCR, LCR, and NASBA. Preferably, the amplification method is PCR. The PCR (polymerase chain reaction) is well known in the art. In general, PCR is a method for amplifying a nucleic acid, which includes annealing, i.e., binding a primer to a complementary template using a reaction solution containing a pair of primers, a template, polymerase and dNTP at annealing temperature, performing polymerization starting from the attached primer at polymerization temperature, denaturing polymerized double-stranded nucleic acids at denaturation temperature and repeating the above procedures. The reaction solution for the amplification depends on the type of the amplification method. However, in general, the reaction solution may be any solution in which a nucleic acid may be polymerized by polymerase. In an exemplary embodiment of the present invention, the reaction solution is a PCR reaction solution, which is conventionally used in the art.

In addition, the mixture of the nucleic acid containing sample and the salt solution may further comprise 0 to 40% of PEG.

The present invention will be described in more detail by presenting examples. These examples are for illustrative purpose, and are not intended to limit the scope of the present invention.

### EXAMPLE

### Example 1

Isolation of Nucleic Acids

In Example 1, DNA was isolated from a DNA containing sample using a solid phase material coated with a carboxyl group or an amino group. A plain glass, a glass coated with an amino group, and a glass coated with a carboxyl group were respectively used as a solid phase material. pBR322 plasmid DNA (about 4.3 kb, available from Promega) was used as the DNA. The isolation procedure was as follows.

1. pBR322 plasmid DNAs were dissolved in distilled water.

2. 100 μl of the pBR322 plasmid DNA (DNA, 1 μg) solution in distilled water was mixed with 100 μl of a 2.5 M NaCl solution containing 20% PEG.

3. 180 μl of the mixture was injected into a polymer chamber so that the mixture came into contact with a glass substrate coated with a carboxyl group or an amino group in the polymer chamber. The polymer chamber has an inlet and an outlet for a sample and a space of 1.6 mm×1.6 mm×0.4 mm and was manufactured by attaching a chamber housing to the glass substrate.

4. After injection, the mixture was incubated at room temperature for 5 minutes and then removed from the polymer chamber.

5. The chamber was washed by injecting a solution containing 70% ethanol and 10 mM EDTA into the chamber and the washing was repeated three times.

6. 180 μl of distilled water was injected into the chamber to elute the attached DNAs from the substrate and the eluted solution was collected.

7. The presence or absence of the DNA was confirmed by an agarose gel electrophoresis for the collected product.

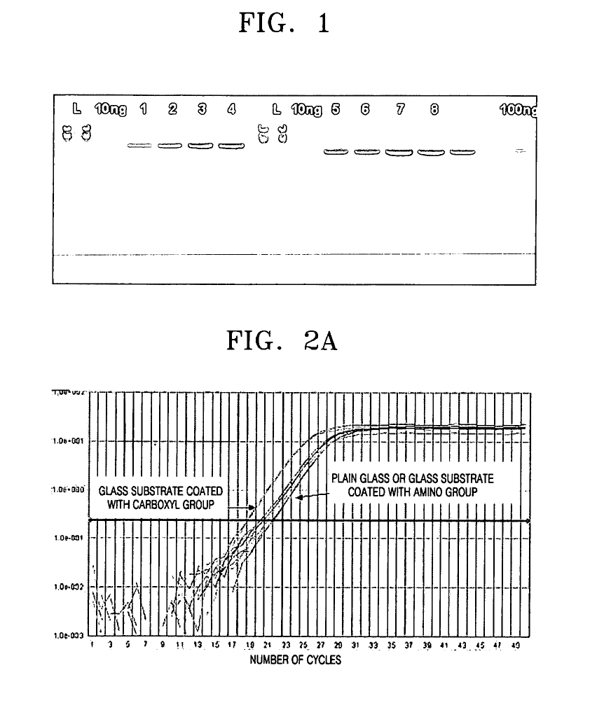

The results are shown in FIG. 1. Referring to FIG. 1, lanes 1 and 2 indicate the results obtained by using a plain glass, lanes 3 and 4 indicate the results obtained by using a glass coated with an amino group (NH₂), lanes 5 and 6 indicate the results obtained by using a glass coated with a carboxyl group (COOH), and lanes 7 and 8 indicate the results obtained by using magnetic particles coated with a carboxyl group (Dynabead™, available from DynaL Biotech). Lanes “10 ng” and “100 ng” indicate a positive control, respectively. As illustrated in FIG. 1, it was confirmed that DNA can be qualitatively isolated on all the tested substrates.

### Example 2

Isolation of HBV Plasmid DNA

In Example 2, to confirm the difference according to the surface property of a substrate, plasmid DNA was isolated on the respective substrates and a real-time PCR was performed using the isolated plasmid DNA as a template. Isolation yields on the respective substrates were compared with one another.

A plain glass, a glass coated with an amino group, and a glass coated with a carboxyl group were respectively used as a solid phase material. HBV plasmid DNA (about 7.3 kb, ATCC No. 45020D) was used as the DNA. The test procedure was as follows.

1. HBV plasmid DNAs were dissolved in distilled water.

2. 100 μl of the HBV plasmid DNA (DNA, 1 μg) solution in distilled water was mixed with 100 μl of a 2.5 M NaCl solution containing 20% PEG.

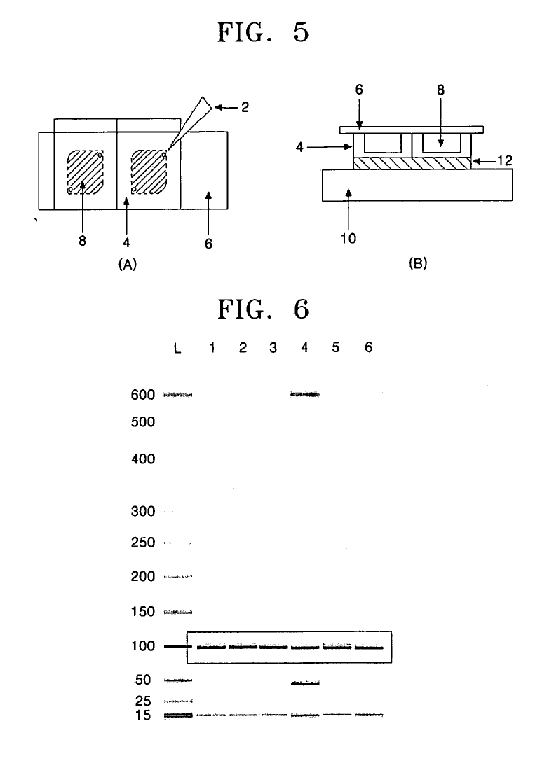

3. 180 μl of the mixture was injected into a polymer chamber so that the mixture came into contact with a glass substrate coated with a carboxyl group or an amino group in the polymer chamber (See, FIG. 5A). The polymer chamber has an inlet and an outlet for a sample and a space of 1.6 mm×1.6 mm×0.4 mm and was manufactured by attaching a chamber housing to the glass substrate.

4. After the injection, the mixture was incubated at room temperature for 5 minutes, and then removed from the polymer chamber.

5. The chamber was washed by injecting a solution containing 70% ethanol and 10 mM EDTA into the chamber and the washing was repeated three times.

6. 180 μl of distilled water was injected into the chamber to elute the attached DNAs from the substrate and the eluted solution was collected.

7. A real-time PCR (ABI7000™) was performed using 100% of the elution solution as a template and using oligonucleotides having SEQ ID NOS. 1 and 2 as primers.

8. After the completion of the PCR, the PCR product was analyzed using an electrophoresis apparatus, Agilent 2100 Bioanalyzer™ (available from Agilent).

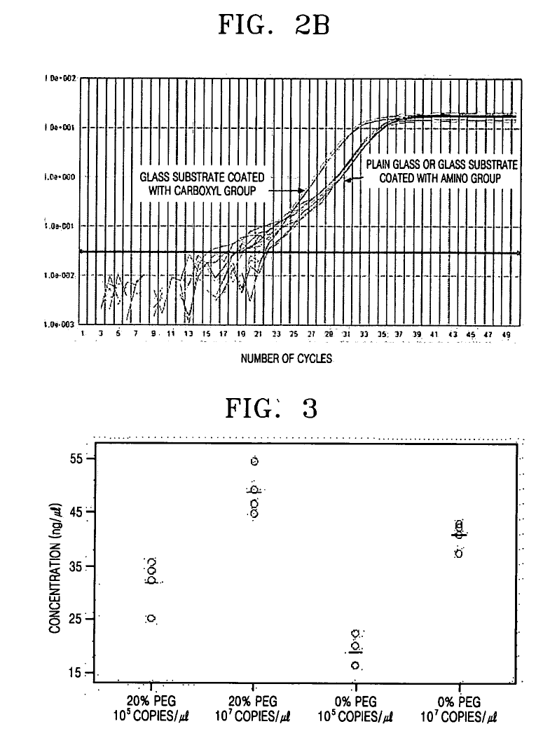

The results are shown in FIGS. 2A and 2B. Referring to FIGS. 2A and 2B, it was confirmed that the DNA product isolated by the method according to an embodiment of the present invention can be used as the template to obtain the PCR product. FIGS. 2A and 2B are views illustrating the results of a real-time PCR for the isolated HBV plasmid DNA products in which the initial concentrations of HBV plasmid DNAs were 10⁷ copies/μl and 10⁵ copies/μl, respectively. The results showed that the highest efficiency of amplification was attained using the glass substrate coated with a carboxyl group. The concentrations of the PCR products after the completion of the real-time PCR are shown in Table 1. Referring to Table 1, the highest concentration of the PCR product was attained using the glass substrate coated with a carboxyl group.

TABLE 1
Concentrations of real-time PCR products obtained by
using the isolated DNA product as a template

| | | Initial concen- | |
|---|---|---|---|
| | | tration of | |
| | | HBV plasmid | |
| | | DNA (copy/μl), | Concentration of |
| | | the volumetric | PCR product |
| Substrate | Binding buffer | amount used | (50 cycles)(ng/μl) |
| Glass | 20% PEG + | 10⁵, 100 μl | 10 |
| | 2.5 M NaCl, | | |
| | 100 μl | | |
| Glass coated | 20% PEG + | 10⁵, 100 μl | 20 |
| with an amino | 2.5 M NaCl, | | |
| group | 100 μl | | |
| Glass coated | 20% PEG + | 10⁵, 100 μl | 25 |
| with a carboxyl | 2.5 M NaCl, | | |
| group | 100 μl | | |

### Example 3

Effects of the Concentration of PEG and the Initial Concentration of DNA on the Efficiency of Isolation of HBV Plasmid DNA

Examples 1 and 2 showed that the substrate coated with a carboxyl group exhibited the highest efficiency of isolation. In Example 3, the effects of the concentration of PEG and the initial concentration of HBV plasmid DNA in a binding buffer on the efficiency of isolation of HBV plasmid DNA were examined.

A glass coated with a carboxyl group was used as a solid phase material.

HBV plasmid DNA (about 7.3 kb, ATCC No. 45020D) was used as the DNA. The test procedure was as follows.

1. HBV plasmid DNAs were dissolved in distilled water.

2. 100 μl of the HBV plasmid DNA (DNA, 1 μg) solution in distilled water was mixed with 100 μl of a 2.5 M NaCl solution containing 20% PEG or no PEG, respectively.

3. 180 μl of the mixture was injected into a polymer chamber so that the mixture came into contact with a glass substrate coated with a carboxyl group in the polymer chamber. The polymer chamber has an inlet and an outlet for a sample and a space of 1.6 mm×1.6 mm×0.4 mm and was manufactured by attaching a chamber housing to the glass substrate (See, FIG. 5A).

4. After the injection, the mixture was incubated at room temperature for 5 minutes, and then removed from the polymer chamber.

5. The chamber was washed three times by injecting a solution containing 70% ethanol and 10 mM EDTA into the chamber.

6. 180 μl of distilled water was injected into the chamber to elute the attached DNAs from the substrate, and the eluted solution was collected.

7. A real-time PCR (ABI7000™) was performed using 100 μl of the elution solution as a template and using oligonucleotides having SEQ ID NOS. 1 and 2 as primers.

8. After the completion of the PCR, the PCR product was analyzed using an electrophoresis apparatus, Agilent 2100 Bioanalyzer™ (available from Agilent).

Referring to FIG. 3, isolation was performed using the binding buffers having the initial concentrations of HBV plasmid DNA of 10⁵ copies/μl and 10⁷ copies/μl, respectively, with the NaCl solution containing 20% PEG or no PEG. Then, the isolated products were subject to a PCR and the concentrations of the PCR products obtained were illustrated in FIG. 3. The binding buffer containing PEG produced a higher concentration of the PCR product than that of the binding buffer containing no PEG.

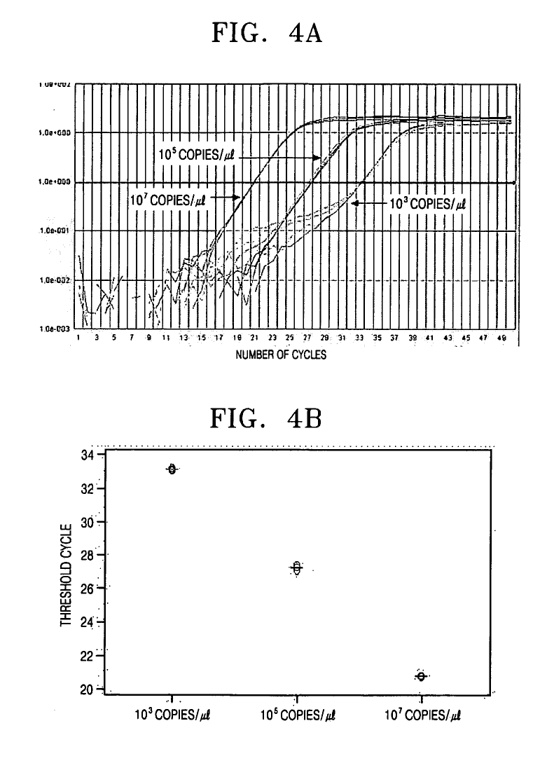

FIGS. 4A and 4B are views illustrating the results of a real-time PCR in which the products isolated using the initial DNA concentrations of 10³, 10⁵ and 10⁷ copies/μl, respectively, were used as a template. FIG. 4A illustrates the results of a real-time PCR and a threshold cycle versus the initial DNA concentrations in FIG. 4A. The threshold cycle means the number of PCR cycles at which the detection signal intensity rises above the threshold value. Referring to FIG. 4B, when the initial concentrations of DNA were 10³, 10⁵ and 10⁷ copies/μl, the threshold cycles were 33.1, 27.3 and 20.8 cycles in average, respectively. That is, there was a difference of about 6.7 cycles between the given concentrations. Generally, in theory, a 10-fold difference in the initial concentration of DNA used as a template in a real-time PCR corresponds to 3.3 cycle difference in the threshold cycle. Thus, in this Example, a difference of about 6.7 cycles is estimated corresponding to about 100-fold difference in the initial DNA concentration. Thus, it was confirmed that when using the method of isolation according to an embodiment of the present invention, the concentration of DNA in the final isolated product is proportional to the initial DNA concentration, implying the constant efficiency of isolation.

### Example 4

PCR on a glass Substrate Coated with a Carboxyl Group

In Example 4, nucleic acids were added to a glass substrate coated with a carboxyl group, and after an PCR was performed on the same substrate, whether the PCR was performed in the glass substrate coated with a carboxyl group was confirmed.

A glass coated with a carboxyl group was used as a solid phase material. A polymer chamber which has an inlet and an outlet for a sample and a space of 1.6 mm×1.6 mm×0.4 mm and was manufactured by attaching a chamber housing to the substrate was used as a chamber for polymerization reaction. HBV plasmid DNA (about 7.3 kb, ATCC No. 45020D) was used as the DNA. The procedure of test was as follows.

1. HBV plasmid DNAs were dissolved in distilled water.

2. 100 μl of the HBV plasmid DNA solution in distilled water was mixed with 100 μl of a buffer solution for a PCR containing oligonucleotides having SEQ ID NOS. 1 and 2 as primers.

3. 180 μl of the mixture was injected into a polymer chamber so that the mixture came into contact with a glass substrate coated with a carboxyl group in the polymer chamber. The polymer chamber has an inlet and an outlet for a sample and a space of 1.6 mm×1.6 mm×0.4 mm and was manufactured by attaching a chamber housing to the glass substrate (See, FIG. 5A).

4. After the injection, the inlet and the outlet were sealed with a polymer cover.

5. The substrate equipped with a chamber housing and containing the mixture of the DNA sample with the PCR solution was turned over and mounted on a heating block in a PCR apparatus (See, FIG. 5B).

Referring to FIGS. 5A and 5B, the polymer chamber used in an embodiment of the present invention is briefly described, as follows: A polymer chamber housing 4 having an inlet and an outlet for a fluid and a space of 1.6 mm×1.6 mm×0.4 mm is attached to a glass substrate 6 to manufacture a polymer chamber 8, and the manufactured polymer chamber 8 is used as a chamber for the isolation of DNA and polymerization reaction (See, FIG. 5A). In FIG. 5A, the DNA sample is injected through the sample inlet into the polymer chamber 8 using a micropipette 2. FIG. 5A is a perspective top view illustrating the polymer chamber housing 4 attached to the substrate 6. FIG. 5B is a sectional view illustrating the polymer chamber 8 mounted on the heating block 10 so as to perform the PCR in the polymer chamber 8. In FIG. 5B, the polymer chamber 8 was connected with the heating block 10 via optical tape 12 (available from ABI). Thus, heat can be transferred to the polymer chamber 8 through the heating block 10 so that thermal cycling can be performed.

6. A PCR was performed using oligonucleotides having SEQ ID NOS. 1 and 2 as primers. The conditions of cycles were 40 cycles of 95° C. for 20 sec, 58° C. for 30 sec and 72° C. for 40 sec using MJ Research PTC-100 apparatus™.

7. After the completion of PCR, the PCR product (about 100 bp) was analyzed by an electrophoresis apparatus, Agilent 2100 Bioanalyzer™ (available from Agilent).

The results of the gel electrophoresis analysis for the PCR product are shown in FIG. 6. As illustrated in FIG. 6, it was confirmed that PCR may be performed on the glass substrate coated with a carboxyl group. The respective lanes in FIG. 6 indicate the results of repeated experiments in the same conditions, showing that the desired PCR products having a size of about 100 bp were reproducibly produced in this Example.

Thus, it was confirmed that the isolation and amplification of nucleic acids can be performed on the same glass substrate coated with a carboxyl group according to an embodiment of the present invention.

According to an embodiment of the present invention, nucleic acids can be efficiently isolated by using a solid phase material coated with a carboxyl group or an amino group, without using a chaotropic material. In addition, nucleic acids can be amplified on the same substrate as used in isolating the nucleic acids.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method for amplifying a nucleic acid using a solid phase material coated with a carboxyl group or an amino group, comprising:
contacting a mixture of a nucleic acid containing sample and a salt solution with the solid phase material coated with a carboxyl group or an amino group to form a nucleic acid-solid phase material complex;
washing the nucleic acid-solid phase material complex with a wash buffer; and
adding a reaction solution for amplifying a nucleic acid to the nucleic acid-solid phase material complex to perform an amplification reaction.

2. The method of claim 1, wherein the nucleic acid containing sample is a biological material.

3. The method of claim 1, wherein the salt is at least one selected from the group consisting of NaCl, MgCl₂, KCl, and CaCl₂.

4. The method of claim 3, wherein the salt is contained in a concentration of 0.5 to 5 M in the salt solution.

5. The method of claim 1, wherein the solid phase material is glass, silicon, polyethylene, polypropylene, polyacrylate or polyurethane.

6. The method of claim 1, wherein the washing the nucleic acid-solid phase material complex is carried out with a wash buffer containing ethanol and EDTA.

7. The method of claim 1, wherein the mixture of the nucleic acid containing sample and the salt solution comprises 0 to 40% of PEG.

8. The method of claim 1, wherein the amplification reaction is a PCR (polymerase chain reaction).


SEQUENCE LISTING (2 sequences)

SEQ ID NO: 1 - DNA, 23 bp, Artificial Sequence
  forward primer
gctttggggcatggacattgacc

SEQ ID NO: 2 - DNA, 23 bp, Artificial Sequence
  reverse primer
agcagaggcggtgtcgaggagat